(19) United States
(12) Patent Application Publication
(10) Pub. No.: US 2004/0174497 A1
(43) Pub. Date: Sep. 9, 2004

(54) Method and system for controlling the movement of a device

(76) Inventor: Manish Sharma, Mountain View, CA (US)

Correspondence Address: HEWLETT-PACKARD DEVELOPMENT COMPANY, Intellectual Property Administration, P.O. Box 272400, Fort Collins, CO, 80527-2400, US

(21) Appl. No.: 10/384,410

(22) Filed: Mar. 7, 2003

(51) Int. Cl. A61B 3/14

(52) U.S. Cl. 351/210

(57) ABSTRACT

A method and system for controlling the movement of a device is disclosed. According to the present invention, a method and system includes moving a device based on the detection of ocular movement. Through the use of the method and system in accordance with the present invention, a user has the ability to automatically control the movement of a device by simply moving her eyes in the direction that she wants the device to move. The method and system include capturing a plurality of images of an ocular unit, determining a direction of movement of the ocular unit based on the plurality of images and moving the device based on the direction of movement of the ocular unit.

### FIELD OF THE INVENTION

The present invention relates to the field of ocular tracking, and more particularly to a method and system for controlling the movement of a device.

### BACKGROUND OF THE INVENTION

Equipment that can monitor the eye movements of a person in response to certain visual stimuli is well known. Typically, the subject would be exposed to a visual stimulus and his ocular reactions recorded by a monitoring apparatus. Such an apparatus can include a light source, visible or infrared, which is reflected off the eye into a suitable detector. The detected signal is then electronically processed to obtain a reading of the eye position at any given time.

Many applications exist for such an apparatus. These include medical diagnosis, military uses such as weapons aiming, training equipment such as aircraft simulators, sports analysis for improving visual techniques and concentration, advertisement testing, design planning as for an automobile dashboard, and testing for visual impact as of highway and store signs. In some of the listed applications, medical diagnosis and aircraft simulators for example, the eye-movement-monitoring apparatus is stationary as is the equipment for presenting the visual stimuli, such as a video monitor. Since the latter two are fixed, the viewer is also stationary. Typically, the subject is seated and his head fixed in place by a chin rest or a bit plate. However, in some applications, the exposure to the requisite stimuli requires movement. Thus, if analysis of a baseball batter's vision as he watches a pitched ball is desired, it would be preferable to actually do that in a batter's box in a realistic situation. Likewise, in advertising applications a subject may be requested to walk down a supermarket aisle so that his response to the most eye-catching containers can be recorded. Stationary equipment obviously cannot accomplish such tasks.

Head-mounted eye-movement-monitoring equipment has been devised which obviates the need to keep the person's head fixed. Since the equipment is affixed to the subject's head, it moves with his head and provides an accurate signal regardless of how he moves it. Such devices have been used in, for example, military applications where head movement is essential (e.g. the helmet of a pilot) and even in applications where head movement is not essential but preferable. As regards the latter, a fixed position for the head is to be avoided when the monitoring session is relatively lengthy because the subject is likely to experience considerable discomfort after awhile and a commensurate decrease in concentration.

The above described eye movement monitoring technology has been limited in its applications due to various limitations in computer processing technology. Essentially, the speed at which computer processors could process the transmitted signals, limited the applications in which this technology could be applied. However, advancements in computer processing technology have greatly increased the speeds at which data can be effectively processed and utilized.

Accordingly, what is needed is a method and system that allows eye movement monitoring technology to take advantage of the advancements in computer processing technology. The method and system should be simple, cost effective and capable of being easily adapted to existing technology. The present invention addresses these needs.

### SUMMARY OF THE INVENTION

The present invention includes a method and system for controlling the movement of a device. According to the present invention, a method and system includes moving a device based on the detection of ocular movement. Through the use of the method and system in accordance with the present invention, a user has the ability to automatically control the movement of a device by simply moving her eyes in the direction that she wants the device to move.

A first aspect of the present invention includes a method for controlling the movement of a device. The method includes capturing a plurality of images of an ocular unit, determining a direction of movement of the ocular unit based on the plurality of images and moving the device based on the direction of movement of the ocular unit.

Another aspect of the present invention includes a system for controlling the movement of a device. The system comprises an image capturing device configured to capture a plurality of images of an ocular unit, a control module coupled to the image capturing device for receiving a plurality of images of an ocular unit wherein the control module is capable of determining a direction of movement of the ocular unit based on the captured plurality of images and a device coupled to the control module wherein the device is configured to move based on signals received from the control module regarding the direction of movement of the ocular unit.

Other aspects and advantages of the present invention will become apparent from the following detailed description, taken in conjunction with the accompanying drawings, illustrating by way of example the principles of the invention.

### BRIEF DESCRIPTION OF THE DRAWINGS

FIG. 1 is a high-level flow chart of a method in accordance with an embodiment of the present invention.

FIG. 2 is a block diagram of a CCD camera system that could be utilized in conjunction with an embodiment of the present invention.

FIG. 3 is a block diagram of an exemplary system in accordance with an embodiment of the present invention.

FIG. 4 is a block diagram of a camera that could be utilized in conjunction with a system in accordance with an embodiment of the present invention.

FIG. 5 is a more detailed block diagram of the CPU of a camera being utilized in conjunction with an embodiment of the present invention.

FIG. 6 is a diagram of a system in accordance with an alternate embodiment of the present invention.

FIG. 7 shows a block diagram of a system in accordance with an alternate embodiment of the present invention.

FIG. 8 shows a block diagram of a computer system that could be utilized in conjunction with an embodiment of the present invention.

FIG. 9 shows a flowchart of a method in accordance with an alternate embodiment of the present invention.

### DETAILED DESCRIPTION

The present invention relates to a method and system for controlling the movement of a device. The following description is presented to enable one of ordinary skill in the art to make and use the invention and is provided in the context of a patent application and its requirements. Various modifications to the preferred embodiment and the generic principles and features described herein will be readily apparent to those skilled in the art. Thus, the present invention is not intended to be limited to the embodiment shown but is to be accorded the widest scope consistent with the principles and features described herein.

The present invention includes a method and system for controlling the movement of a device. According to the present invention, a method and system includes moving a device based on the detection of ocular movement. The present invention takes advantage of advancements in computer processing technology that have greatly increased the speeds at which data can be effectively processed and utilized. Through the use of the method and system in accordance with the present invention, a user has the ability to automatically control the movement of a device by simply moving her eyes in the direction that she wants the device to move.

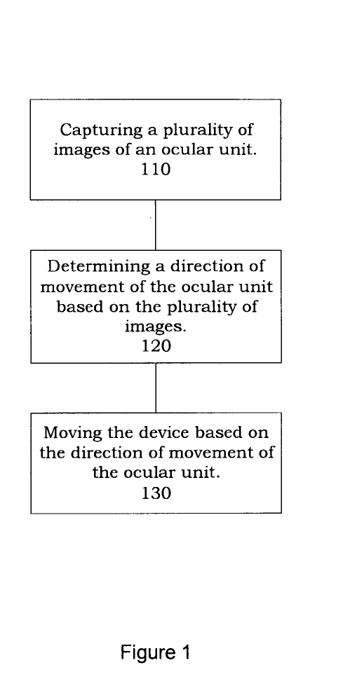

For a further understanding of the present invention, please refer now to FIG. 1. FIG. 1 is a flowchart of a method in accordance with an embodiment of the present invention. A first step 110 includes capturing a plurality of images of an ocular unit. In an embodiment, the ocular unit can be a human eye. The next step 120 includes determining a direction of movement of the ocular unit based on the plurality of images. The final step 130 includes moving a device based on the direction of movement of the ocular unit.

In an embodiment, step 110 is achieved by utilizing an image capturing device. The image capturing device should be capable of capturing multiple images of the ocular unit in a rapid fashion. This could be accomplished with a small Charge Coupled Device (CCD) camera. A CCD is an electronic memory that can be charged by light. CCDs can hold a variable charge, which is why they are used in cameras and scanners to record variable shades of light. CCDs are analog, not digital, and are made of a special type of MOS transistor.

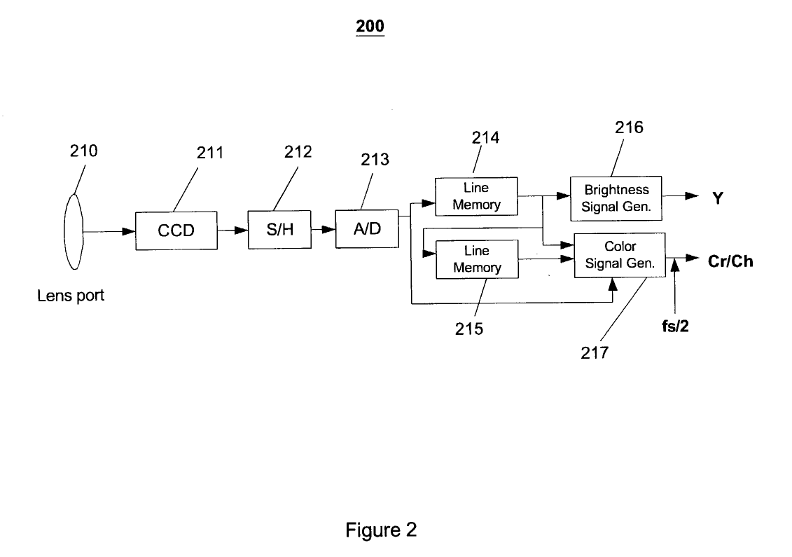

For an example of a CCD camera system that could be utilized in conjunction with the present invention please refer now to FIG. 2. FIG. 2 is a block diagram of a CCD camera system 200 that could be utilized in conjunction with an embodiment of the present invention. As shown in FIG. 2, the conventional CCD camera system 200 includes a lens part 210 for focusing the optical signals of an object, a CCD 211 for converting the imaged optical signals into electrical signals when the optical signals from the lens part 210 is imaged, a sampling/holding device 212 for carrying out a sampling/holding function, so as to remove unnecessary signals such as noise and the like from among the output video signals of the CCD 211 and an analog-digital converter 213 for converting the output analog video signals of the sampling/holding device 212 into digital video signals, so as to carry out digital signal processing.

The system 200 further includes a first line memory 214 for storing the one period (1H) delayed output signals of the analog-digital converter 213, a second line memory 215 for storing the one period (1H) delayed signals of the first line memory 214, a brightness signal generator 216 for generating brightness signals Y by using the stored signals of the first line memory 214 and a color signal generator 217 for generating color signals Cr and Cb by utilizing an internal color difference signal matrix and by receiving the output signals of the analog-digital converter 213 and the stored signals of the first and second line memories 214 and 215.

The conventional CCD camera system 200 as described above operates in the following manner. In processing color signals by using a single plate type CCD, if the color is to be restored, independent color components have to be provided rather than just color components from a tingle pixel. Recently, among methods using a single plate CCD, a complementary filtering method (a filtering method using the color components of magenta Mg, cyan Cy, yellow Ye, green G) has been used because of its superior spectrum sensitivity characteristics.

The color filter array pattern of the single plate type CCD is constituted such that, horizontally, there are repeatedly arranged lines S1 having components “magenta+cyan” and “green+cyan”, and lines S2 having components “green+yellow” and “magenta+yellow”. Vertically, if it is assumed that the components “magenta+cyan” and “green+yellow” are Nth line pixels, then the components “green+cyan” and “magenta+yellow”are (N−1)th or (N+1)th line pixels. The single plate CCD is further broken down vertically into odd fields and even fields, and the pixel components of the lines S1 and S2 are different according to their respective fields.

As described above, the color filter array of the CCD has a sequential structure for each pixel and for each line, and therefore, if the color signals of red R, green G and blue B are to be generated, horizontal and vertical interpolation processes have to be carried out by utilizing the adjacent pixels of the color filter array. Particularly, if the vertical interpolation is to be carried out, the two line memories 214 and 215 of FIG. 2 are used, so as to store the signals of the currently inputted video signals which are delayed by one period (1H) and which are delayed by two periods (2H). Then, based on the signals delayed by one period, an interpolation is carried out by using the currently inputted video signals and the signals delayed by two periods (2H).

Referring back to FIG. 1, in an embodiment, step 120 can be accomplished utilizing image analysis techniques on the captured images. Utilizing image analysis, the captured images of the ocular unit can be analyzed and the direction of movement of the ocular unit can be determined.

In an embodiment, step 130 can be accomplished utilizing a control module coupled to the device for controlling the movement of the device once the direction of movement of the ocular unit has been determined. In an embodiment, the device being controlled is a digital camera or the like. In an alternate embodiment, the device being controlled is a cursor on a computer screen.

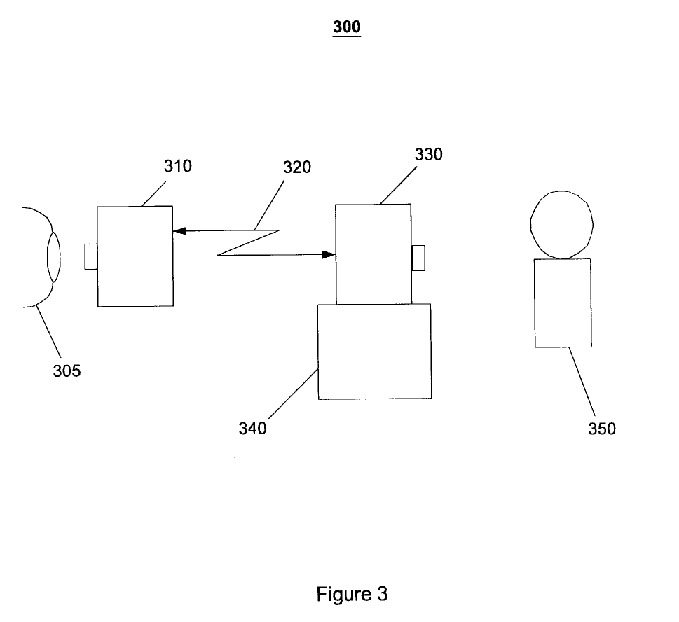

FIG. 3 shows an exemplary system 300 in accordance with an embodiment of the present invention. The system 300 includes an image capturing device 310 and a camera 330. The camera 330 is coupled to the image capturing device 310 via a communication link 320 and can be placed on a stand 340 in front of an object 350. In accordance with this embodiment, the ocular unit is a human eye 305 and the image capturing device 310 is positioned in front of the eye 305. The image capturing device 310 captures a plurality of images of the eye 305 and sends these images to the camera 330 via the communication link 320. The image analysis software within the camera 330 is then implemented on the captured images to determine the direction of movement of the eye 305. Finally, the camera 330 is configured to “move” based on the direction of movement of the eye 305.

Additionally, eye movements such as blinking can be used to mimic button presses to activate/control the camera 330. For example, the camera 330 could be configured to snap a picture of the object 350 every time the user blinks.

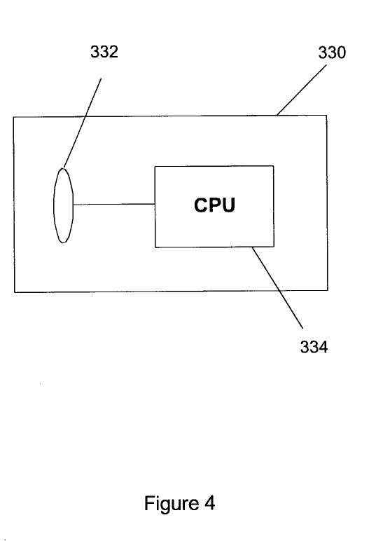

For an example of a camera 330 that could be utilized in conjunction with the present invention please refer now to FIG. 4. FIG. 4 is a block diagram of a camera 330 in accordance with an embodiment of the present invention. The camera 330 includes a lens 332 that is coupled to a central processing unit (CPU) 334. The CPU 334 typically includes a conventional processor device for controlling the operation of the camera 330. The CPU 334 can be capable of concurrently running multiple software routines and modules to control the various process of the camera. The CPU 334 is coupled to an I/O interface 336 for allowing communications to and from the CPU 334. For example, I/O interface 336 provides for communications to and from image capturing device 310.

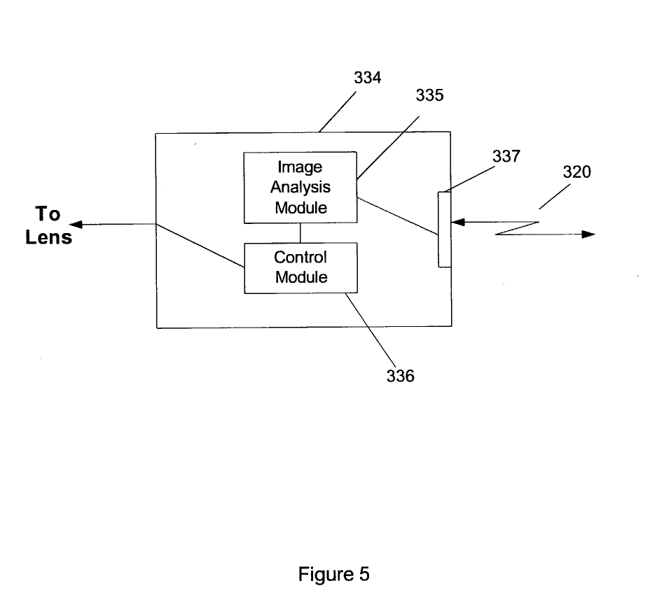

In an embodiment, the CPU 334 includes a control module and an image analysis module. For a better understanding, please refer to FIG. 5. FIG. 5 is a more detailed block diagram of the CPU 334 of the camera 330 being utilized in conjunction with an embodiment of the present invention. As can be seen in FIG. 5, the CPU 334 includes an image analysis module 335, a control module 336 and an I/O interface 337 wherein the control module 336 is coupled to image analysis module 335. The image analysis module 335 receives data from the communication link 320 via the I/O interface 337.

Accordingly, the image analysis module 335 receives captured images via the communication link 320 and determines the direction of movement of the eye 305. The image analysis module 335 then transmits this information to the control module 336 whereby the control module 336 moves the lens 332 based on the information received from the image analysis module 335.

Although the above described embodiment is described as being utilized in conjunction with a camera that takes still pictures, one of ordinary skill in the art will readily recognize that the present invention could be utilized in conjunction with a video camera or a variety of other cameras while remaining within the spirit and scope of the present invention. For example, eye movements can be utilized in conjunction with the present invention to start or stop recording on a video camera.

Referring back to FIG. 3, the image capturing device 310 could be configured to capture images a predetermined rate. For example, the image capturing device 310 could be configured to capture images of the eye 305 at a rate of 1 image every second, 1 image every 2 seconds, etc. However, because it takes a normal human brain roughly 1/10 of a second to process an image, the image capturing device 310 should not be configured to capture images at a rate faster than 10 images per second.

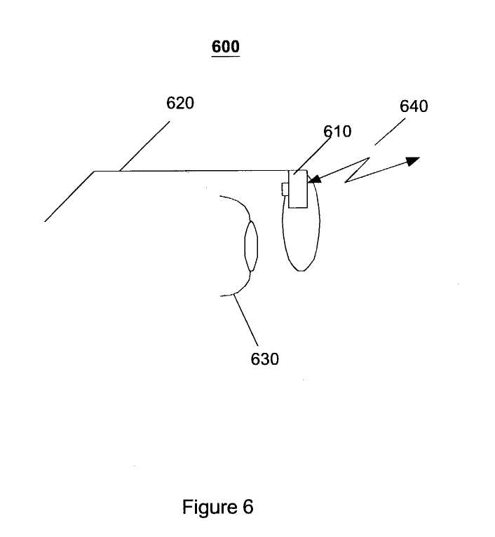

In an embodiment, the image capturing device 310 is a small CCD camera capable of being mounted on a pair of eyeglasses. FIG. 6 is an illustration of an alternate embodiment of the present invention. Accordingly, FIG. 6 shows a small CCD camera 610 mounted on a pair of eyeglasses 620 wherein the camera 610 is positioned to capture images of a user's eye 630 for the purpose of determining the direction of movement of the eye 630. The camera 610 can be coupled to another device (not shown) via communication link 640 whereby the movement of the device can be controlled based on the direction of movement of the eye 630.

The communication link (320, 640) could be a cable link or a wireless link. In accordance with an embodiment of the present invention, the communication link is a radio link in accordance with the Bluetooth Global Specification for wireless connectivity. Bluetooth is an open standard for short-range transmission of digital voice and data between mobile devices (laptops, PDAs, phones) and desktop devices. It supports point-to-point and multipoint applications. Unlike Infra-Red, which requires that devices be aimed at each other (line of sight), Bluetooth uses omni-directional radio waves that can transmit through walls and other non-metal barriers. Bluetooth transmits in the unlicensed 2.4 GHz band and uses a frequency hopping spread spectrum technique that changes its signal 1600 times per second. If there is interference from other devices, the transmission does not stop, but its speed is downgraded.

The Bluetooth baseband protocol is a combination of circuit and packet switching. Each data packet is transmitted in a different hop frequency wherein the maximum frequency hopping rate is 1600 hops/s. Bluetooth can support an asynchronous data channel, up to three simultaneous synchronous voice channels, or a channel which simultaneously supports asynchronous data and synchronous voice. Each voice channel supports 64 kb/s synchronous (voice) link. The asynchronous channel can support a symmetric link of maximally 721 kb/s in either direction while permitting 57.6 kb/s in the return direction, or a 432.6 kb/s symmetric link.

Although the above described embodiments of the present invention are described as being utilized to control the movement of a camera, one of ordinary skill in the art will readily recognize that the features of the present invention could be implemented to control the movement of a variety of devices while remaining within the spirit and scope of the present invention. For example, an alternate embodiment of the present invention could include a personal computer system whereby the movement of the cursor on the computer screen could be controlled based on the movement of the eye.

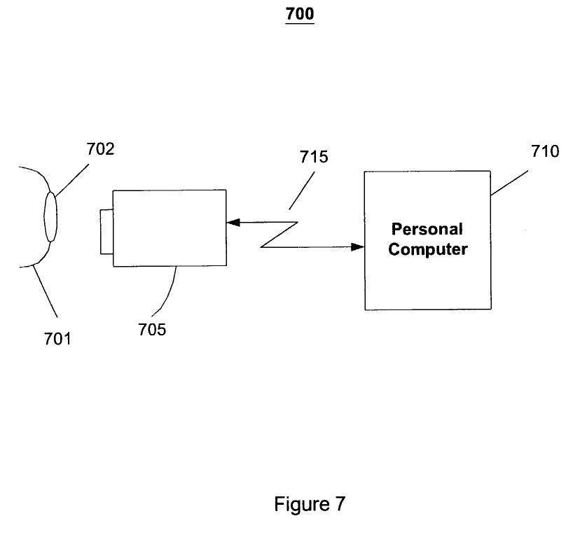

FIG. 7 shows a system 700 in accordance with an alternate embodiment of the present invention. The system 700 includes an image capturing device 705 coupled to a computer system 710 via a communication link 715. The image capturing device 7Q5 is configured to capture images of an eye 701 for purpose of determining the direction of movement of the eye 701. As previously articulated, the communication link 715 could be implemented via a cable link or a wireless link.

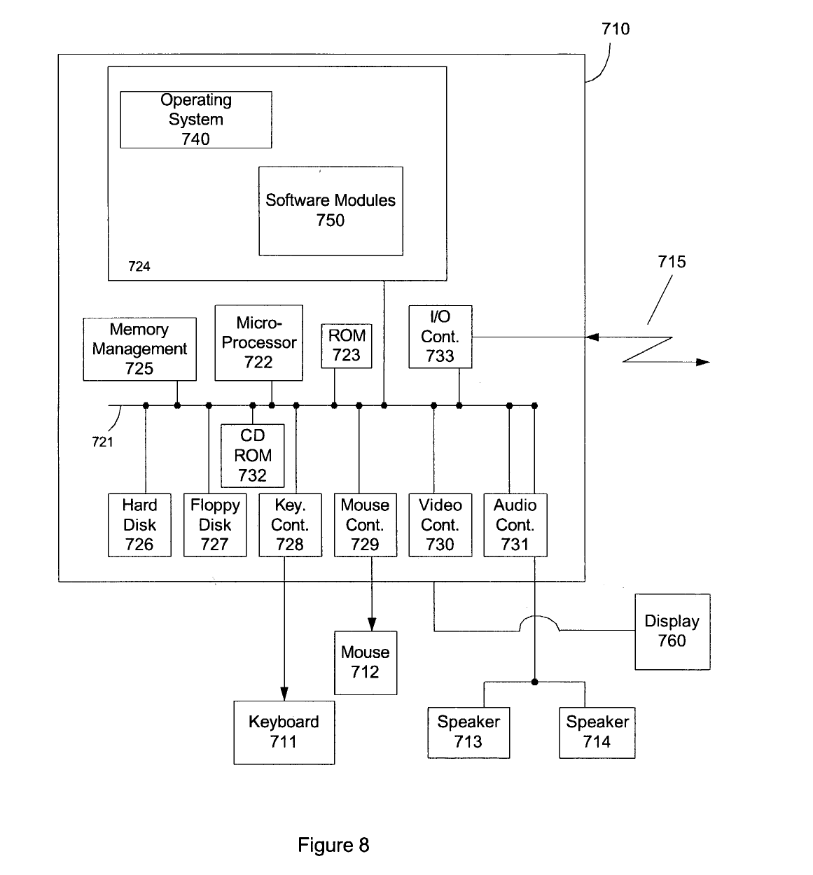

Referring back to FIG. 7, the system 700 can include a PC 710. For an example of such a PC, please refer now to FIG. 8. FIG. 8 is an illustration of a PC 710 that can be utilized in conjunction with the system 700. The PC 710, including, a keyboard 711 and a mouse 712 depicted in block diagram form. The PC 710 includes a system bus or plurality of system buses 721 to which various components are coupled and by which communication between the various components is accomplished. The microprocessor 722 is connected to the system bus 721 and is supported by read only memory (ROM) 723 and random access memory (RAM) 724 also connected to the system bus 721. A microprocessor is one of the Intel family of microprocessors including the 386, 486 or Pentium microprocessors. However, other microprocessors including, but not limited to, Motorola's family of microprocessors such as the 68000, 68020 or the 68030 microprocessors and various Reduced Instruction Set Computer (RISC) microprocessors such as the PowerPC chip manufactured by IBM. Other RISC chips made by Hewlett Packard, Sun, Motorola and others may be used in the specific computer.

The ROM 723 contains, among other code, the Basic Input-Output system (BIOS) which controls basic hardware operations such as the interaction of the processor and the disk drives and the keyboard. The RAM 724 is the main memory into which the operating system 740 and software modules 750 are loaded. The memory management chip 725 is connected to the system bus 721 and controls direct memory access operations including, passing data between the RAM 724 and hard disk drive 726 and floppy disk drive 727. The CD ROM 732 also coupled to the system bus 721 is used to store a large amount of data, e.g., a multimedia program or presentation.

Various I/O controllers are also connected to this system bus 721. These I/O controllers can include a keyboard controller 728, a mouse controller 729, a video controller 730, and an audio controller 731. As might be expected, the keyboard controller 728 can provide the hardware interface for the keyboard 711, the mouse controller 729 can provide the hardware interface for mouse 712, the video controller 730 can provide the hardware interface for the display 760, and the audio controller 731 can provide the hardware interface for the speakers 713, 714.

One of ordinary skill in the art will readily recognize that the PC 710 can include a personal-digital-assistant (PDA), a laptop computer or a variety of other devices while remaining within the spirit and scope of the present invention.

In an embodiment, another I/O controller 733 is coupled to the image capturing device 705 (via communication link 715) and can be configured to control a cursor that is displayed on the display 760. The I/O controller 733 receives captured images of the eye 701 from the image capturing device 705 and the image analysis module 750 analyses the images and determines the direction of movement of the eye 701. Finally, the cursor on the display 760 “moves” based on the direction of movement of the eye 701.

In an embodiment, the image capturing device 705 is mounted on a pair of eyeglasses and sends data to the I/O controller 733 via a cable link or a wireless link. In an alternate embodiment, the image capturing device 705 is mounted on the display 760 and is coupled to the I/O controller 733 via a cable link. Additionally, eye movements such as blinking can be used to mimic mouse clicks or button presses to activate/control images/icons on the display 760. There could be a distinction between involuntary blinks and deliberate blinks whereby a mouse click could be triggered by two quick blinks, one long blink, etc.

In another embodiment, the image capturing device 705 could be utilized to detect the size and depth of the pupil 702 of the eye 701 in order to determine the range of distance at which the eye 701 is presently focusing. Accordingly, in an embodiment where the device being controlled is a camera, the size and depth of the pupil 702 of the eye 701 could be utilized to adjust the focus the lens of the camera.

In an alternate embodiment, a double-imaging system could be incorporated whereby the size and depth of the pupils of each eye is detected by two separate image capturing devices. The focusing distance of the eyes is accordingly determined by analyzing the images captured by the two separate image capturing devices whereby the difference between the images captured by the two separate image capturing devices indicates exactly at what distance the eyes are focusing.

The above-described embodiments of the invention may also be implemented, for example, by operating a computer system to execute a sequence of machine-readable instructions. The instructions may reside in various types of computer readable media. In this respect, another aspect of the present invention concerns a programmed product, comprising computer readable media tangibly embodying a program of machine readable instructions executable by a digital data processor to perform the method in accordance with an embodiment of the present invention.

This computer readable media may comprise, for example, RAM (not shown) contained within the system. Alternatively, the instructions may be contained in another computer readable media such as a magnetic data storage diskette and directly or indirectly accessed by the computer system. Whether contained in the computer system or elsewhere, the instructions may be stored on a variety of machine readable storage media, such as a DASD storage (e.g. a conventional “hard drive” or a RAID array), magnetic tape, electronic read-only memory, an optical storage device (e.g., CD ROM, WORM, DVD, digital optical tape), paper “punch” cards, or other suitable computer readable media including transmission media such as digital, analog, and wireless communication links. In an illustrative embodiment of the invention, the machine-readable instructions may comprise lines of compiled C, C++, or similar language code commonly used by those skilled in the programming for this type of application arts.

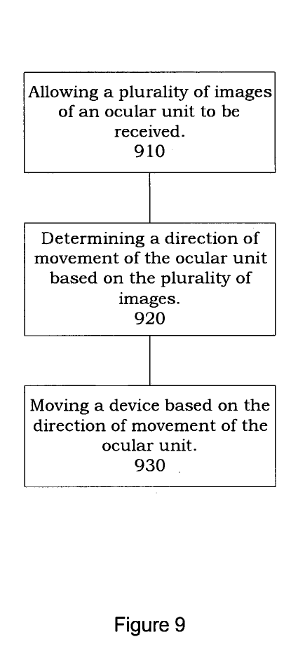

For a better understanding of a method in accordance with an alternate embodiment of the present invention please refer now to FIG. 9. FIG. 9 is a flowchart of program instructions that could be contained on a computer readable medium in accordance with an alternate embodiment of the present invention. A first step 910 involves allowing a plurality of images of an ocular unit to be received. In an embodiment, the ocular unit is a human eye and the images are received from an image capturing device. A second step 920 includes determining a direction of movement of the ocular unit based on the plurality of images. A final step 930 includes moving a device based on the direction of movement of the ocular unit. In an embodiment, the device can be still or video camera. In an alternate embodiment, the device can be a cursor on a computer screen.

A method and system for controlling the movement of a device is disclosed. According to the present invention, a method and system includes moving a device based on the detection of ocular movement. Through the use of the method and system in accordance with the present invention, a user has the ability to automatically control the movement of a device by simply moving her eyes in the direction that she wants the device to move.

Although the present invention has been described in accordance with the embodiments shown, one of ordinary skill in the art will readily recognize that there could be variations to the embodiments and those variations would be within the spirit and scope of the present invention. Accordingly, many modifications may be made by one of ordinary skill in the art without departing from the spirit and scope of the appended claims.

What is claimed is:

1. A method for controlling the movement of a device comprising:
capturing a plurality of images of an ocular unit;
determining a direction of movement of the ocular unit based on the plurality of images; and
moving the device based on the direction of movement of the ocular unit.

2. The method of claim 1 wherein the act of capturing a plurality of images of an ocular unit comprises:
utilizing an image capturing device to capture the plurality of images of the ocular unit.

3. The method of claim 2 wherein the ocular unit comprises a human eye.

4. The method of claim 3 wherein the human eye further comprises a pupil, the device comprises a camera and the image capturing device is utilized to determine the size and depth of the pupil whereby the size and depth of the pupil are utilized to focus the camera.

5. The method of claim 3 wherein the image capturing device comprises a charge coupled device.

6. The method of claim 5 wherein the device comprises another image capturing device.

7. The method of claim 5 wherein the device comprises a cursor.

8. The method of claim 1 wherein the act of determining a direction of movement further comprises:
utilizing image analysis techniques on the plurality of captured images to determine a direction of movement of the ocular unit.

9. The method of claim 8 wherein the act of capturing a plurality of images of an ocular unit further comprises capturing the plurality of images at rate of at most 1 image per 1/10 second.

10. A system for controlling the movement of a device comprising:
means for capturing a plurality of images of an ocular unit;
means for determining a direction of movement of the ocular unit based on the plurality of images; and
means for moving the device based on the direction of movement of the ocular unit.

11. The system of claim 10 wherein the means for capturing a plurality of images of an ocular unit comprises:
means for utilizing an image capturing device to capture the plurality of images of the ocular unit.

12. The system of claim 11 wherein the ocular unit comprises a human eye.

13. The system of claim 12 wherein the image capturing device comprises a charge coupled device.

14. The system of claim 13 wherein the device comprises another image capturing device.

15. The system of claim 13 wherein the device comprises a cursor.

16. The system of claim 10 wherein the means for determining a direction of movement further comprises:
means for utilizing image analysis techniques on the plurality of captured images to determine a direction of movement of the ocular unit.

17. The system of claim 16 wherein the means for capturing a plurality of images of an ocular unit further comprises means for capturing the plurality of images at rate of at most 1 image per 1/10 second.

18. A system for controlling movement of a device:
an image capturing device configured to capture a plurality of images of an ocular unit;
a control module coupled to the image capturing device for receiving a plurality of images of an ocular unit wherein the control module is capable of determining a direction of movement of the ocular unit based on the captured plurality of images; and
a device coupled to the control module wherein the device is configured to move based on signals received from the control module regarding the direction of movement of the ocular unit.

19. The system of claim 18 wherein the ocular unit comprises a human eye.

20. The system of claim 19 wherein the image capturing device comprises a charge coupled device.

21. The system of claim 20 wherein the device comprises another image capturing device.

22. The system of claim 20 wherein the device comprises a cursor.

23. The system of claim 17 wherein the control module further comprises:
means for utilizing image analysis techniques on the plurality of captured images to determine a direction of movement of the ocular unit.

24. The system of claim 23 wherein the image capturing device further comprises means for capturing the plurality of images at rate of at most 1 image per 1/10 second.

25. The system of claim 24 wherein the image capturing device is mounted to eyeglasses.

26. A computer readable medium comprising program instructions for controlling the movement of a device, the program instructions comprising the steps of:
allowing a plurality of images of an ocular unit to be received;
determining a direction of movement of the ocular unit based on the plurality of images; and
moving the device based on the direction of movement of the ocular unit.

27. The computer readable medium of claim 26 wherein the plurality of images of the ocular unit are received from an image capturing device.

28. The computer readable medium of claim 27 wherein the ocular unit comprises a human eye.

29. The computer readable medium of claim 28 wherein the determining a direction of movement of the human eye further comprises utilizing image analysis techniques on the plurality of received images to determine a direction of movement of the human eye.

30. A device comprising:
receiving means for receiving a plurality of images of an ocular unit;
determining means for determining a direction of movement of the ocular unit based on the plurality of images; and
moving means coupled to the determining means and the receiving means for moving the device based on the direction of movement of the ocular unit.

31. The device of claim 30 wherein the receiving means and the determining means are included in an image analysis module.

32. The device of claim 31 further comprising a lens coupled to the moving means wherein the moving means comprises a control module.

33. The device of claim 32 wherein moving the device based on the direction of movement of the ocular unit further comprises moving the lens based on the direction of movement of the ocular unit.

34. The device of claim 33 wherein the ocular unit further comprises a human eye wherein the human eye includes a pupil and the image analysis module is utilized to determine the size and depth of the pupil whereby the size and depth of the pupil are utilized to focus the lens.
